(19) United States
(12) Patent Application Publication
(10) Pub. No.: US 2011/0065672 A1
(43) Pub. Date: Mar. 17, 2011

(54) BISPHOSPHONATE-PROSTATIC ACID PHOSPHATASE INHIBITOR CONJUGATES TO TREAT PROSTATE CANCER BONE METASTASIS

(75) Inventors: ALEXANDER KIRSCHENBAUM, NEW YORK, NY (US); ALICE C. LEVINE, NEW YORK, NY (US); STEVEN L. REGEN, ALLENTOWN, PA (US); VACLAV JANOUT, BETHLEHEM, PA (US)

(73) Assignees: MOUNT SINAI SCHOOL OF MEDICINE OF NEW YORK UNIVERSITY, NEW YORK, NY (US); LEHIGH UNIVERSITY, BETHLEHEM, PA (US)

(21) Appl. No.: 12/883,662

(22) Filed: Sep. 16, 2010

Related U.S. Application Data
(60) Provisional application No. 61/243,073, filed on Sep. 16, 2009.

(51) Int. Cl. A61K 31/664; C07F 9/44; A61P 35/00

(52) U.S. Cl. 514/108; 558/155

(57) ABSTRACT

The present invention concerns conjugate compounds comprising a bisphosphonate covalently bonded to a prostatic acid phosphatase inhibitor and compositions comprising such conjugates. Methods for treating and inhibiting prostate cancer bone metastases, and determining whether a conjugate is useful for such treatment are also provided. In some instances, the bisphosphonate is alendronate, and it is covalently bonded to either tartaric acid or glyceric acid.

### FIELD OF THE INVENTION

The present invention relates to compositions for selectively targeting bone tissue, and for treating or inhibiting prostate cancer bone metastasis.

### BACKGROUND OF THE INVENTION

Prostate cancer has a propensity to spread to bone. Unfortunately, there are currently no curative therapies for prostate cancer bone metastasis. Both normal and cancerous bone remodeling relies upon dynamic interactions and balance between osteoclasts (bone cells that remove bone tissue by removing mineralized matrix), osteoblasts (cells responsible for bone formation) and the bone matrix. The initial step in normal bone remodeling and prostate cancer bone targeting is thought to involve activated osteoclasts that produce bone acid phosphatase. Bone acid phosphatase (also known as tartrate-resistant acid phosphatase) is the enzyme that degrades bone matrix. Once prostate cancer cells interact with bone osteoclasts and bone matrix is degraded, stored growth factors in the matrix as well as those produced by osteoblasts stimulate prostate cancer cell growth in the bone microenvironment. FIG. 1 illustrates the possible growth factors and cytokines that play a role in the cycle of prostate cancer-bone cell interactions (Guise et al. (2006). Clinical Cancer Research 12 (supplement 20), 6213s-6216s).

Prostatic acid phosphatase (PAP), a phosphotryosyl protein phosphatase, is a prostate epithelium-specific secretory protein that is found in large amounts in the seminal fluid. High PAP levels have been found in patients having prostate cancer metastatic to bone, and consequently, PAP has been used as a human tumor marker (Gutman et al. (1936). American Journal of Cancer, 28, 485-495). PAP has subsequently been used as a marker for the response of prostate cancer bone metastases to hormonal therapy (Huggins and Hodges (1941). Cancer Research, 1, 293-297). Although PAP is useful as a prostate cancer tumor marker, prostate specific antigen (PSA) has largely replaced PAP in this role. Similar to bone acid phosphatase, PAP can alter the bone microenvironment, making it more conducive for a tumor to spread.

Bisphosphonates such as alendronic acid (sold as Fosamax® by Merck) and risedronic sodium (sold as Actonel® by Proctor & Gamble) are currently utilized to treat osteoporosis and to reduce the morbidity (pain, fractures) due to prostate cancer bone metastasis. Bisphosphonates exhibit a high affinity to the bone mineral hydroxyapatite, and accumulate minimally at other sites in the body. Consequently, these bisphosphonates have also been used as carriers for therapeutic agents to bone for the treatment of arthritis and bone metastasis (Gittens et al. (2005). Advanced Drug Delivery Reviews, 57 1011-1036). A bisphosphonate that is conjugated to a PAP inhibitor such as tartrate may selectively target bone tissue to inhibit the secretion of PAP and reduce and prevent both bone complications and cancer cell growth in bone.

### SUMMARY OF THE INVENTION

The present invention is directed to conjugate compounds comprising a bisphosphonate covalently bonded to a PAP inhibitor, pharmaceutically acceptable salts thereof, and compositions comprising the same. The bisphosphonate can be bonded directly to the PAP inhibitor or alternatively, through a linker.

Non-limiting examples of bisphosphonates useful in the conjugates of the invention include, for example, pamidronate, neridronate, olpadronate, alendronate, ibandronate, risedronate or zoledronate. In a preferred embodiment, the bisphosphonate is alendronate.

Non-limiting examples of PAP inhibitors useful in the conjugates of the invention include, for example, a hydroxycarboxylic acid (e.g., tartaric acid, glyceric acid, citric acid, lactic acid, glycolic acid, malic acid, or tartronic acid), an oxoanion (e.g., vanadate, molybdate, or tungstate) and a heteropolyanion (e.g., a heteropolymolybdate, a heteropolytungstate, a heteropolyoxometalate, or a heteropolyperiodate). In a preferred embodiment, the PAP inhibitor is a hydroxycarboxylic acid. In a further preferred embodiment, the hydroxycarboxylic acid is tartaric acid or glyceric acid.

In a preferred embodiment, the conjugate of the invention comprises bisphosphonate alendronate which is covalently bonded to PAP inhibitor tartaric acid or glyceric acid.

The present invention is also directed to methods of preventing, treating or inhibiting a prostate cancer bone metastasis with a conjugate compound comprising a bisphosphonate and a PAP inhibitor. The method comprises administering an effective amount of a conjugate compound comprising a bisphosphonate covalently bonded to a PAP inhibitor to a subject in need of prostate cancer bone metastasis treatment. The route of administration can be oral or parenteral (e.g., intravenous).

The present invention also provides a method for making a PAP inhibitor orally active. In one embodiment, the method comprises covalently bonding the PAP inhibitor to a bisphosphonate. In a preferred embodiment, the bisphosphonate is alendronate and the PAP inhibitor is selected from tartaric acid and glyceric acid.

### BRIEF DESCRIPTION OF THE FIGURES

FIG. 1 is a diagram showing certain growth factors and cytokines that play a role in the cycle of prostate cancer-bone cell interactions.

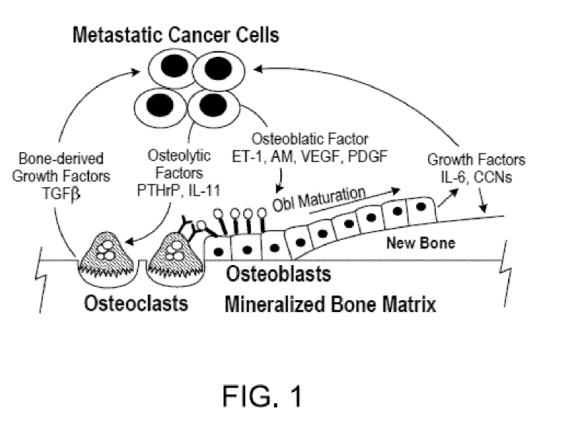

FIG. 2 shows representative immunohistochemical stains of a prostate cancer bone metastases sample from a patient who received androgen ablation. The sample was stained from the presence of hematoxylin and eosin (panel A), nuclear androgen receptor expression (panel B), prostate specific antigen (panel C) and prostate acid phosphatase (panel D).

FIG. 3 shows immunohistochemical stains of a mouse tibia bone sample that was harvested from an immunocompromised mouse inoculated with VCaP prostate cancer cells. Panel A is an H & E stain, panel B is a prostatic acid phosphatase stain, and panel C is a prostate specific antigen stain.

FIG. 4 shows three bar graphs showing the effect of tartrate on MC3T3 and VCaP cell growth (panel A); secretion of prostatic acid phosphatase (panel B); and secretion of alkaline phosphatase (panel C).

FIG. 5 is a graph showing the effect of tartrate and two bisphosphonate-PAP inhibitor conjugates on PAP secretion.

FIG. 6 shows two bar graphs demonstrating the effect of tartrate on RAW cell growth determined by hemacytometer (panel A) and differentiation determined by the mean number of multinucleated cells estimated by multiple field counts in 20× objective view (panel B).

### DETAILED DESCRIPTION OF THE INVENTION

PAP, secreted by human prostate cancer cells, may be active in the acid environment of bone, acting similarly to osteoclast-derived bone acid phosphatase to degrade bone matrix and release growth factors. However, the two enzymes are distinguishable because PAP is inhibited by tartrate (see U.S. Pat. No. 5,763,490), while bone acid phosphatase is not. Thus, inhibiting PAP, for example with tartrate, may serve to inhibit tumor growth in bone, by interrupting the cycle in which prostate tumor cells stimulate bone cells to produce growth factors. Additionally, combining the PAP inhibitor with the bone targeting drug bisphosphonate may serve to selectively target bone cells with the PAP inhibitor, thereby allowing for oral or parenteral administration of a prostate cancer bone metastasis drug. The conjugate may prevent and/or treat a prostate cancer bone metastasis by the mechanism described above.

### DEFINITIONS

A “bisphosphonate,” as used herein, has the following core structure:

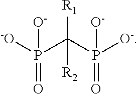

The bisphosphonates provided in Table 1 can be used in preparing the conjugates of the present invention.

TABLE 1
Non-limiting list of bisphosphonates for use with the present invention

| Bisphosphonate | | |
|---|---|---|
| 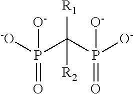 | R₁ side chain | R₂ side chain |
| Etidronate | 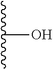 | 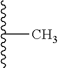 |
| Clodronate | 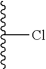 | 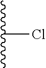 |
| Tiludronate | 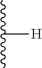 | 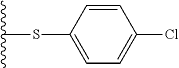 |
| Pamidronate | 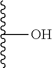 | 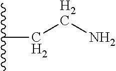 |
| Neridronate | 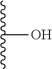 | 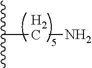 |
| Olpadronate | 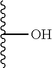 | 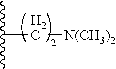 |
| Alendronate | 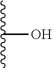 | 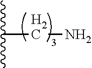 |
| Ibandronate | 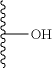 | 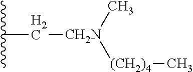 |
| Risedronate | 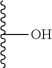 | 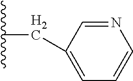 |
| Zoledronate | 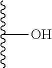 | 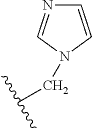 |

A “hydroxycarboxylic acid,” as used herein, refers to a carboxylic acid with an additional hydroxyl group. Therefore, a dicarboxylic acid falls under the definition of a “hydroxycarboxylic acid.” Examples of hydroxycarboxylic acids useful for the present invention include tartaric acid, glyceric acid, citric acid, lactic acid, glycolic acid, malic acid, threonic acid, tartronic acid, malonic acid, glutaric acid, pimelic acid and adipic acid. A reference to a hydroxycarboxylic acid includes salts and esters of the hydroxycarboxylic acid (e.g., a reference to tartaric acid includes tartrate salts).

“PAP,” as used herein, refers to prostatic acid phosphatase.

“ALP,” as used herein, refers to bone alkaline phosphatase.

The terms “subject” and “patient” are used interchangeably to refer to an experimental or veterinary animal (e.g., mouse, rat, rabbit, dog, cat) or to a human.

“Effective amount” refers to an amount of a bisphosphonate conjugate of the present invention sufficient to result in a desired result. The response can be, for example, inhibition of PAP secretion from prostate cancer cells, inhibition of PAP activity, inhibition or prevention of prostate cancer bone metastases, or inhibition of tumor growth. Additionally or alternatively, the desired result can be an attenuation of bone alkaline phosphatase secretion from osteoblast cells, or a decrease in osteoblast cell growth.

### Conjugate Compounds Comprising a Bisphosphonate and a PAP Inhibitor

In certain aspects, the present invention is directed to compounds comprising a bisphosphonate covalently bonded to a PAP inhibitor. The bisphosphonate can be, for example, a compound provided in Table 1, above.

In certain embodiments, the PAP inhibitor of the invention is tartaric acid or a salt or ester thereof, i.e., a tartrate. However, other PAP inhibitors are contemplated for use with the present invention (see, e.g., Kilsheimer and Axelrod (1957) JBC 227 879-890). For example, hydroxycarboxylic acids such as glyceric acid, citric acid, lactic acid, glycolic acid, malic acid, threonic acid and tartronic acid, and esters and salts thereof, may be used in the conjugates of the present invention.

Hydroxycarboxylic acid derivatives (e.g., tartaric acid derivatives) can be conjugated to a bisphosphonate such as alendronate to arrive at a conjugate of the present invention. For example, a carboxylic group present in the tartaric acid can be reacted with an alcohol or aromatic alcohol such as phenol or naphthol, to form phenyl or naphthyl derivatives, respectively. In addition, a hydroxyl group in a hydroxycarboxylic acid such as tartaric acid can be reacted with acids like benzoic acid or 1-naphthyl acetic acid to form an ester linkage. PAP binding may be improved with such derivatives because the derivatives that contain an aromatic functional group can be bound in a hydrophobic pocket located in the binding region of PAP (see U.S. Pat. No. 5,763,490).

Other compositions that may be useful as PAP inhibitors are inorganic oxoanions like vanadate, molybdate and tungstate. In addition, heteropolyanions, which inhibit PAP may also be used. Examples of these are: heteropolymolybdates, heteropolytungstates, heteropolyoxometalates and heteropolyperiodates. Heteropolyoxometalate complexes useful for the present invention include: [C(NH₂)₃]2[(CH₃)₂AsMo₄O₁₅H], (Bu₄N)₂(CH₃)₂AsMo₄O₁₅H, (Bu₄N)₂ (C₆H₅)₂AsMo₄O₁₅H, (Bu₄N)₂Mo₈O₂₈, (NH₄)₆MO₇O₂₄.4H₂O, (NH₄)₃FeMo₆O₂₄H₆.6H₂O, (NH₄)₄GeMo₁₂O₂₄H₆.xH₂O, (NH₄)₈ThMo₁₂O₄₂.7H₂O, (NH₄)₆AS₂MO₁₈O₆₂.xH₂O.

Although conjugation of a bisphosphonate to a PAP inhibitor can be through direct covalent attachment, linkers joining the two moieties may also be employed. For example, N-(2-hydroxypropyl)methacrylamide (HPMA) can be used to link a bisphosphonate to a PAP inhibitor. Other linkers that can be employed in compounds of the present invention include polyethylene glycol, carboxylic acids and dicarboxylic acids (e.g., succinic acid).

In two preferred embodiments of the invention, the PAP inhibitor is tartaric acid (or a salt or ester thereof) or glyceric acid (or a salt or ester thereof) and each is covalently bonded to alendondrate, as set forth in Examples 1 and 2, below.

In certain embodiments, the bisphosphonates disclosed in Table 1 are each directly bonded to tartrate to provide ten distinct conjugate compounds of the present invention. In other embodiments, each of the bisphosphonates in Table 1 is bonded directly to glyceric acid to arrive at ten additional conjugate compounds of the present invention.

In some embodiments, the specific conjugates recited above include a linker (such as polyethylene glycol) between the PAP inhibitor and bisphosphonate.

### Characterization of the Bisphosphonate-PAP Inhibitor Conjugates of the Present Invention

Once a bisphosphonate-PAP inhibitor conjugate has been made, it can be characterized in a number of ways. For example, the conjugate can added to a prostate cancer bone metastasis cell preparation, and the preparation can be stained for the presence of prostatic acid phosphatase. The stain can be compared to a sample that has not been treated with the particular conjugate.

Alternatively or additionally, a conjugate of the present invention can be orally or parenterally administered to a subject known to have a prostate cancer bone metastasis. The metastasis, or portion thereof, can be harvested and subjected to immunohistochemical staining for prostatic acid phosphatase. A different subject known to have prostate cancer bone metastasis can have a conjugate injected directly into the metastasis site. The difference between PAP expression in the two subjects gives an indirect measure of how well the bisphosphonate targets the metastasis site, when administered either orally or parenterally.

The conjugates of the present invention can also be added to osteoblast/prostate cancer cell co-cultures in vitro. If a particular conjugate is effective, PAP will be inhibited which will attenuate osteoblast growth. Therefore, the number of osteoblasts can be counted before and after addition of the conjugate to determine the effectiveness of the conjugate.

ELISA assays can also be employed to determine the amount of PAP secretion from prostate cancer cells (either single culture or co-culture with osteoblast cells), before and after addition of a conjugate of the present invention. Additionally or alternatively, PAP enzyme activity assays can be employed to determine whether a conjugate is effective in inhibiting PAP.

Bone alkaline phosphatase (ALP) is secreted by pre-osteoblast cells (e.g., MC3T3 cells), and is correlated with pre-osteoblast differentiation into osteoblasts cells. Accordingly, ALP secretion can be measured to indirectly determine whether the inhibition of PAP secretion by a conjugate of the present invention also serves to attenuate pre-osteoblast differentiation.

Salts, Solvates, Stereoisomers, Derivatives of the Compounds of the Invention

The methods of the present invention further encompass the use of salts, solvates, and stereoisomers of the bisphosphonate-PAP inhibitor conjugates disclosed above.

Typically, a pharmaceutically acceptable salt of a bisphosphonate-PAP inhibitor conjugate of the present invention is prepared by reaction of the conjugate with a desired acid or base as appropriate. The salt may precipitate from solution and be collected by filtration or may be recovered by evaporation of the solvent. For example, an aqueous solution of an acid such as hydrochloric acid may be added to an aqueous suspension of the bisphosphonate-PAP inhibitor conjugate and the resulting mixture evaporated to dryness (lyophilized) to obtain the acid addition salt as a solid. Alternatively, the bisphosphonate-PAP inhibitor conjugate may be dissolved in a suitable solvent, for example an alcohol such as isopropanol, and the acid may be added in the same solvent or another suitable solvent. The resulting acid addition salt may then be precipitated directly, or by addition of a less polar solvent such as diisopropyl ether or hexane, and isolated by filtration.

The acid addition salts of the bisphosphonate-PAP inhibitor conjugates may be prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

Pharmaceutically acceptable base addition salts are formed with metals or amines, such as alkali and alkaline earth metals or organic amines. Examples of metals used as cations are sodium, potassium, magnesium and calcium. Examples of suitable amines are N,N′-dibenzylethylenediamine, chloroprocaine, diethanolamine, dicyclohexylamine, ethylenediamine, N-methylglucamine, and procaine.

The base addition salts of said acidic compounds are prepared by contacting the free acid form with a sufficient amount of the desired base to produce the salt in the conventional manner. The free acid form may be regenerated by contacting the salt form with an acid and isolating the free acid.

Those skilled in the art of organic chemistry will appreciate that many organic compounds can form complexes, i.e., solvates, with solvents in which they are reacted or from which they are precipitated or crystallized, e.g., hydrates with water. The salts of compounds of the present invention may form solvates such as hydrates. Techniques for the preparation of solvates are well known in the art (see, e.g., Brittain. Polymorphism in Pharmaceutical Solids. Marcel Decker, New York, 1999).

### Compositions of the Invention

The conjugates used herein may be formulated for administration in any convenient way for use in human or veterinary medicine and the invention therefore includes within its scope pharmaceutical compositions comprising a compound of the invention adapted for use in human or veterinary medicine. Such compositions may be presented for use in a conventional manner with the aid of one or more suitable carriers. Acceptable carriers for therapeutic use are well-known in the pharmaceutical art, and are described, for example, in Remington's Pharmaceutical Sciences, Mack Publishing Co. (A. R. Gennaro, 1985). The choice of pharmaceutical carrier can be selected with regard to the intended route of administration and standard pharmaceutical practice. The pharmaceutical compositions may comprise as, in addition to, the carrier any suitable binder(s), lubricant(s), suspending agent(s), coating agent(s), and/or solubilizing agent(s).

Further, a composition of the present invention can contain two or more distinct conjugate compounds. For example, a composition can include both N-Alendronyl-D-Glyceramide and N-Alendronyl-L-Tartaric Acid Monamide. In another embodiment, the composition can include two conjugates each having a distinct bisphosphonate moiety.

Preservatives, stabilizers, dyes and even flavoring agents may be provided in the pharmaceutical composition. Examples of preservatives include sodium benzoate, ascorbic acid and esters of p-hydroxybenzoic acid. Antioxidants and suspending agents may be also used.

The compounds used in the invention may be milled using known milling procedures such as wet milling to obtain a particle size appropriate for tablet formation and for other formulation types. Finely divided (nanoparticulate) preparations of the compounds may be prepared by processes known in the art, for example see International Patent Application No. WO 02/00196 (SmithKline Beecham).

The compounds and pharmaceutical compositions of the present invention can be administered orally (e.g., as a tablet, sachet, capsule, pastille, pill, boluse, powder, paste, granules, bullets or premix preparation, ovule, elixir, solution, suspension, dispersion, gel, syrup or as an ingestible solution). Additionally, the conjugates presented herein can be formulated for parenteral administration (e.g., intravenous, intramuscular, intraarticular, subcutaneous, intradermal, epicutantous/transdermal, transmucosal, and intraperitoneal). Compounds may be present as a dry powder for constitution with water or other suitable vehicle before use, optionally with flavoring and coloring agents. Solid and liquid compositions may be prepared according to methods well-known in the art. Such compositions may also contain one or more pharmaceutically acceptable carriers and excipients which may be in solid or liquid form.

Dispersions can be prepared in a liquid carrier or intermediate, such as glycerin, liquid polyethylene glycols, triacetin oils, and mixtures thereof. The liquid carrier or intermediate can be a solvent or liquid dispersive medium that contains, for example, water, ethanol, a polyol (e.g., glycerol, propylene glycol or the like), vegetable oils, non-toxic glycerine esters and suitable mixtures thereof. Suitable flowability may be maintained, by generation of liposomes, administration of a suitable particle size in the case of dispersions, or by the addition of surfactants.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia.

Additionally, lubricating agents such as magnesium stearate, stearic acid, glyceryl behenate and talc may be included.

Examples of pharmaceutically acceptable disintegrants for oral compositions useful in the present invention include, but are not limited to, starch, pre-gelatinized starch, sodium starch glycolate, sodium carboxymethylcellulose, croscarmellose sodium, microcrystalline cellulose, alginates, resins, surfactants, effervescent compositions, aqueous aluminum silicates and crosslinked polyvinylpyrrolidone.

Examples of pharmaceutically acceptable binders for compositions useful herein include, but are not limited to, acacia; cellulose derivatives, such as methylcellulose, carboxymethylcellulose, hydroxypropylmethylcellulose, hydroxypropylcellulose or hydroxyethylcellulose; gelatin, glucose, dextrose, xylitol, polymethacrylates, polyvinylpyrrolidone, sorbitol, starch, pre-gelatinized starch, tragacanth, xanthane resin, alginates, magnesium-aluminum silicate, polyethylene glycol or bentonite.

Examples of pharmaceutically acceptable fillers for oral compositions include, but are not limited to, lactose, anhydrolactose, lactose monohydrate, sucrose, dextrose, mannitol, sorbitol, starch, cellulose (particularly microcrystalline cellulose), dihydro- or anhydro-calcium phosphate, calcium carbonate and calcium sulfate.

Examples of pharmaceutically acceptable lubricants useful in the compositions of the invention include, but are not limited to, magnesium stearate, talc, polyethylene glycol, polymers of ethylene oxide, sodium lauryl sulfate, magnesium lauryl sulfate, sodium oleate, sodium stearyl fumarate, and colloidal silicon dioxide.

Examples of suitable pharmaceutically acceptable odorants for the oral compositions of the present invention include, but are not limited to, synthetic aromas and natural aromatic oils such as extracts of oils, flowers, fruits (e.g., banana, apple, sour cherry, peach) and combinations thereof, and similar aromas. Their use depends on many factors, the most important being the organoleptic acceptability for the population that will be taking the pharmaceutical compositions.

Examples of suitable pharmaceutically acceptable dyes useful for the compositions of the present invention include, but are not limited to, synthetic and natural dyes such as titanium dioxide, beta-carotene and extracts of grapefruit peel.

Examples of pharmaceutically acceptable coatings useful for the oral compositions of the present invention, typically used to facilitate swallowing, modify the release properties, improve the appearance, and/or mask the taste of the compositions include, but are not limited to, hydroxypropylmethylcellulose, hydroxypropylcellulose and acrylate-methacrylate copolymers.

Suitable examples of pharmaceutically acceptable sweeteners for the oral compositions of the present invention include, but are not limited to, aspartame, saccharin, saccharin sodium, sodium cyclamate, xylitol, mannitol, sorbitol, lactose and sucrose.

Suitable examples of pharmaceutically acceptable buffers useful for the compositions of the present invention include, but are not limited to, citric acid, sodium citrate, sodium bicarbonate, dibasic sodium phosphate, magnesium oxide, calcium carbonate and magnesium hydroxide.

Examples of pharmaceutically acceptable surfactants useful for the oral and parenteral compositions of the present invention include, but are not limited to, sodium lauryl sulfate and polysorbates.

Solid compositions of a similar type may also be employed as fillers in gelatin capsules. Preferred excipients in this regard include lactose, starch, a cellulose, or high molecular weight polyethylene glycols. For aqueous suspensions and/or elixirs, the agent may be combined with various sweetening or flavoring agents, coloring matter or dyes, with emulsifying and/or suspending agents and with diluents such as water, ethanol, propylene glycol and glycerin, and combinations thereof.

Suitable examples of pharmaceutically acceptable preservatives include, but are not limited to, various antibacterial and antifungal agents such as solvents, for example ethanol, propylene glycol, benzyl alcohol, chlorobutanol, quaternary ammonium salts, and parabens (such as methyl paraben, ethyl paraben, propyl paraben, etc.).

Representative examples of pharmaceutically acceptable stabilizers and antioxidants for use in the present invention include, but are not limited to, ethylenediaminetetriacetic acid (EDTA), thiourea, tocopherol and butyl hydroxyanisole.

The pharmaceutical compositions of the invention may contain from 0.01 to 99% weight per volume of the active material (i.e., the bisphosphonate-PAP inhibitor conjugate compound).

### Uses of the Invention

The bisphosphonate-PAP inhibitor of the present invention can be used to treat, inhibit or prevent a prostate cancer bone metastasis or metastases. In one embodiment, an effective amount of a bisphosphonate-PAP inhibitor conjugate, pharmaceutically acceptable salt or composition thereof, is administered to a subject or patient in need of prostate cancer bone metastasis treatment. The composition can either be administered orally or parenterally.

In another embodiment, a method for inhibiting the activity and/or expression of PAP is provided. The method comprises, administering to a subject in need thereof, an effective amount of a bisphosphonate-PAP inhibitor conjugate, pharmaceutically acceptable salt or composition thereof. The composition can either be administered orally or parenterally.

The present invention also provides a method for making a PAP inhibitor orally active. In one embodiment, the method comprises covalently bonding the PAP inhibitor to a bisphosphonate. In a further embodiment, the bisphosphonate is alendronate and the PAP inhibitor is selected from tartaric acid and glyceric acid.

The bisphosphonate-PAP inhibitor conjugates of the present invention can also be used to attenuate or prevent prostate cancer cells from residing in bone from acting like osteoclast-derived bone acid phosphatase (i.e., degrading bone matrix, thereby setting up a PCa-bone vicious cycle).

The conjugates of the present invention may serve a duel purpose. Bisphosphonates are currently used to reduce morbidity (pain, fractures) due to metastasis of prostate cancer to bone. Accordingly, the conjugate compounds of the present invention may reduce both bone complications such as pain and fractures, as well as reduce cancer cell growth in bone (i.e., by inhibiting prostatic acid phosphatase).

The present invention is further illustrated by reference to the Examples below. However, it should be noted that these Examples, like the embodiments described above, are illustrative and are not to be construed as restricting the enabled scope of the invention in any way.

### EXAMPLES

### Example 1

### Synthesis of N-Alendronyl-D-Glyceramide

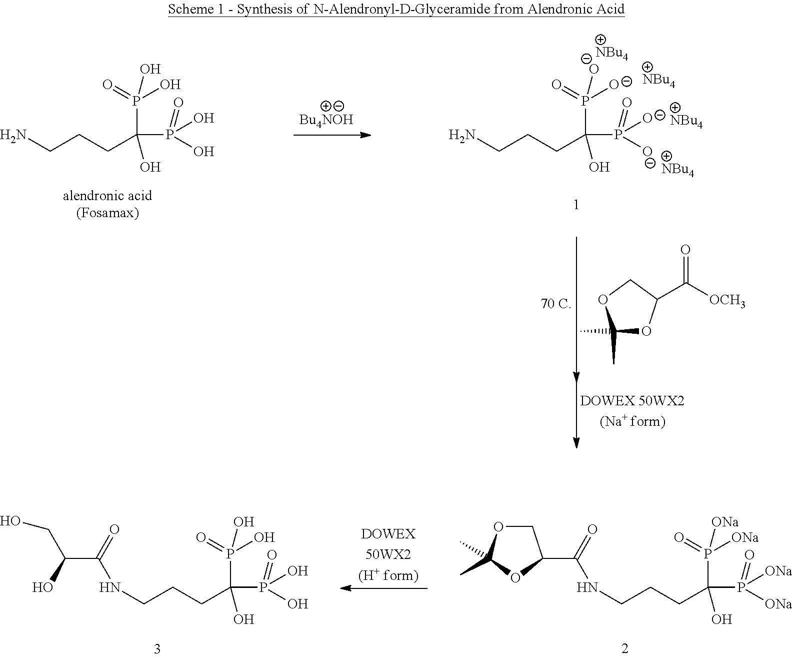

Alendronic acid (62.0 mg, 0.25 mmol) was added to a solution of 800 mg of Bu₄N⁺OH⁻.30H₂O (Aldrich) in 0.20 mL of water. After the mixture was homogenized by vortex mixing at 40° C. for 30 min, the water was removed by freeze drying for 24 h to give 0.306 mg (101%) of (4-Amino-1-hydroxy-butylidene)bisphosphonic acid, N,N,N,N-tetrabutyl-ammonium salt (Tetrabutylammonium alendronate), (1) as a white solid having ¹H NMR (500 MHz, D₂O, 23° C.): 3.10 (m, 32H), 2.53 (t, 2H), 1.77 (m, 2H), 1.67 (m, 2H), 1.54 (m, 32H), 2.26 (m, 32H), 0.85 (t, 48H).

A solution of 0.306 mg (0.25 mmol) of tetrabutylammonium alendronate in 0.630 g of methyl-2,3-O-isopropylidene-D-glycerate (3.93 mmol) was stirred at 70° C. for 72 h under an argon atmosphere. After cooling the reaction mixture to room temperature, 5 mL of CH₂Cl₂ was added. The resulting solution was extracted three times with 2 mL of water and the aqueous extracts combined and washed two times with CH₂Cl₂. The aqueous solution was then added to 400 mg of wet (H₂O) ion exchange resin DOWEX 50W X2, Na⁺ form, (Supelco) and gently shaken at room temperature for 1 h. The aqueous phase was separated from resin and freeze dried to give 0.112 mg of crude N-Alendronyl-2,3-O-isopropylidene-D-glyceramide tetrasodium salt (2) (see scheme 1). This product was then purified by HPLC using a 7.8×300 mm Nova-Pak HR RP C18 column and an eluent that consisted of CH₃OH/H₂O (30/70, v/v) under isocratic conditions with a flow rate of 1.5 mL/min. Fractions having a retention time of 5.2-5.9 min were collected, and the solvent removed under reduced pressure (10 Torr, 30° C.), followed by freeze drying for 24 h to give 70.8 mg of (2) having ¹H NMR (500 MHz, D₂O, 23° C.): 4.65 (m, 1H), 4.36 (m, 1H), 4.09 (m, 1H), 3.28 (t, 2H), 1.93 (m, 2H), 1.84 (m, 2H), 1.50 (s, 3H), 1.44 (s, 3H).

N-Alendronyl-2,3-O-isopropylidene-D-glyceramide, (2) (26.5 mg) was dissolved in 1.0 mL of water and stirred at 50° C. with 10 mg of an acidic form of DOWEX™ 50WX2 for 4 h. Prior to use, the DOWEX™ resin was washed five times with 10 mL of water. Deprotection of the vicinal diol was monitored by ¹H NMR by following the disappearance of isopropylidene moiety and the appearance of acetone. After complete deprotection, the reaction mixture was freeze dried for 24 h to give 15.3 mg of N-Alendronyl-D-glyceramide (3) having ¹H NMR (500 MHz, D₂O, 23° C.): 4.04 (m, 1H), 3.61 (m, 2H), 3.12 (m, 2H), 1.82 (m, 2H), 1.66 (m, 2H); HRMS for C₇H₁₆NO₁₀P₂ ([M-H]⁻) calcd: 336.0255. found: 336.0251.

### Example 2

### Synthesis of N-Alendronyl-L-Tartaric Acid Monamide

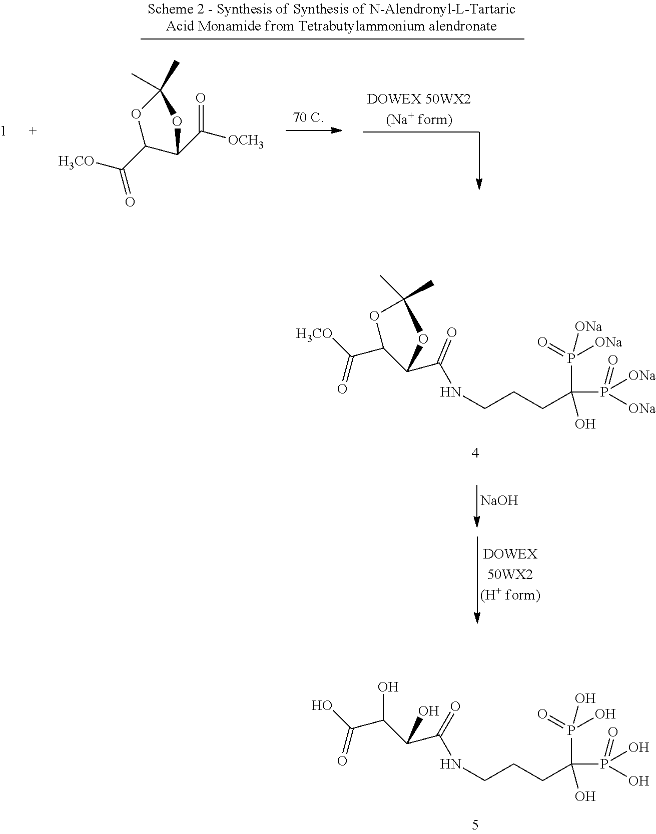

A solution of 0.306 mg (0.25 mmol) of tetrabutylammonium alendronate (1) in 1.200 g of dimethyl-2,3-O-isopropylidene-L-tartarate (5.5 mmol) was stirred at 70° C. for 72 h under an argon atmosphere. After cooling the reaction mixture to room temperature and adding 5 mL of CH₂Cl₂, the mixture was extracted three times with 2 mL of water. The aqueous extracts were combined and washed twice with 2 mL of CH₂Cl₂. The resulting aqueous solution was then added to 400 mg of wet (H₂O) ion exchange resin DOWEX™ 50W X2, Na⁺ form, (Supelco) and gently shaken for 1 h at room temperature. The aqueous phase was separated from the DOWEX™ resin, and freeze dried to give 0.117 mg of crude N-Alendronyl-2,3-β-isopropylidene-L-tartaramidomethylester tetrasodium salt (4). This product was then purified by HPLC using a 7.8×300 mm Nova-Pak HR RP C18 column and an eluent that consisted of CH₃OH/H₂O (30/70, v/v) under isocratic conditions with a flow rate of 1.5 mL/min. Fractions having a retention time of 5.0-5.8 min. were collected, and the solvent removed under reduced pressure (10 Torr, 30° C.), followed by freeze drying for 24 h to give 70.0 mg of (4) having ¹H NMR (500 MHz, D₂O, 23° C.): 4.80 (d, 1H), 4.73 (d, 1H), 3.81 (s, 3H), 3.28 (t, 2H), 1.74-1.93 (m, 4H), 1.48 (s, 3H), 1.43 (s, 3H).

N-alendronyl-2,3-O-isopropylidene-L-tartaramidomethylester tetrasodium salt (4) (35 mg) was saponified using 0.6 mL 0.3 M NaOH by heat for 6 h at 50° C. The progress of the saponification was monitored via ¹H NMR by following the disappearance of the absorbance of the methyl ester at 3.8 ppm and the appearance of CH₃OH at 3.3 ppm. 250 mg of the acidic form of DOWEX™ 50WX2 resin was added to the product mixture (prior to use, the DOWEX™ resin was washed five times with 10 mL of water). The mixture and resin combination was then heated at 50° C. for 4 h.

The deprotection of the vicinal diol was monitored by ¹H NMR by following the disappearance of the isopropylidene structure and appearance of acetone. The mixture was then freeze dried for 24 h to give 21.3 mg of N-Alendronyl-L-tartaric acid monoamide (5) having ¹H NMR (500 MHz, D₂O, 23° C.): 3.42 (s, 1H), 3.39 (s, 1H), 3.12 (m, 2H), 1.67-1.79 (m, 4H); HRMS for C₈H₁₆NO₁₂P₂ ([M-H]⁻) calcd: 380.0153. found: 380.0146.

### Example 3

### Immunohistochemical Stains of Bone Metastatic Prostate Cancer Samples

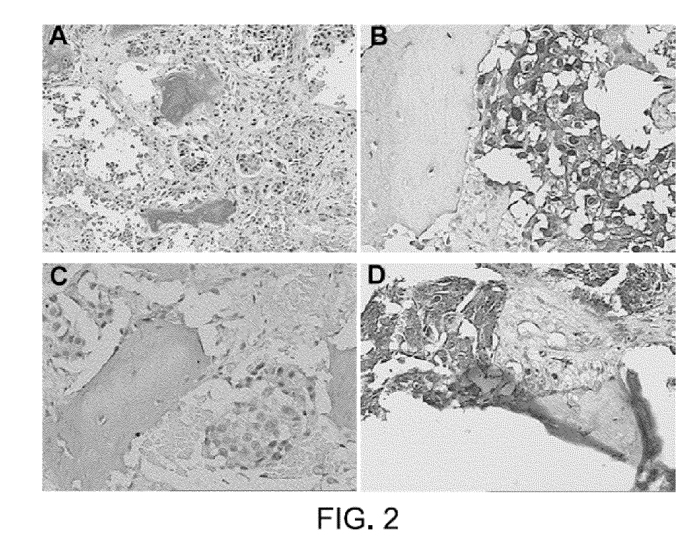

To determine the degree of prostatic acid phosphatase expression in patients who have bone metastatic prostate cancer, human prostate cancer bone metastases derived from 7 patients (i.e., n=7) were immunostained for the expression of androgen receptor (AR), prostate-specific antigen (PSA) and prostatic acid phosphatase (see Table 2 and FIG. 2). Although AR and PSA expression was heterogeneous in these advanced metastatic lesions, each specimen showed uniform expression of prostatic acid phosphatase.

TABLE 2
Summary of immunohistochemical staining for PSA
and PAP in 7 patients (4 of 7 had androgen ablative
therapy prior to surgery on bone metastases).

| Tissue Type | PSA | Prostatic Acid Phosphatase |
|---|---|---|
| Primary Ca (n = 7) | +++ | +++ |
| Lymph Node (n = 7) | ++ | +++ |
| Bone Met (n = 3) | ++ | +++ |
| Bone Met (Androgen Ablated) | + | +++ |
| (n = 4) | | |

FIG. 2 shows representative immunohistochemical stains of a prostate cancer bone metastasis sample from a patient who received androgen ablation therapy. Each of the four samples were stained for the presence of hematoxylin and eosin (H & E, panel A in FIG. 2), nuclear androgen receptor expression (AR, panel B in FIG. 2), prostate specific antigen (PSA, panel C in FIG. 2) and prostate acid phosphatase (PAP, panel D in FIG. 2). The dark, uniform staining in panel D indicates positive immunohistochemical staining for prostatic acid (PAP) in bone metastases as compared to no evidence of expression of PSA (Panel C). These results indicate that PAP is expressed in prostate cancer bone metastases.

### Example 4

### Mouse Model for Prostate Cancer Bone Metastasis

### In Vivo Model System

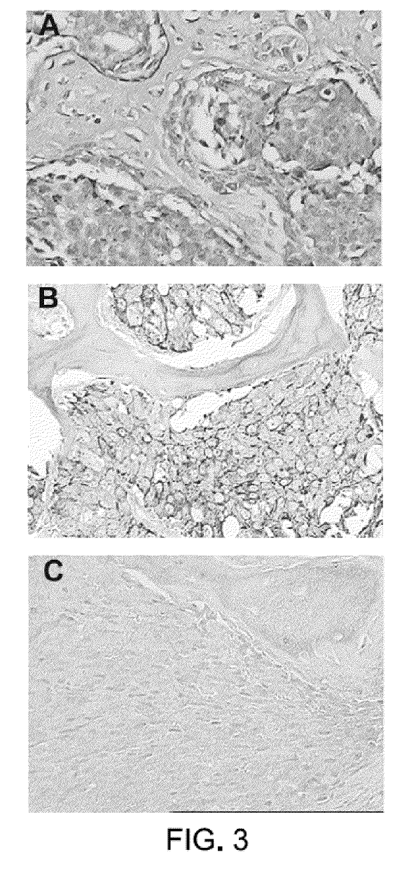

In order to study the behavior of human prostate cancer cells in bone in an animal model, VCaP cells (human origin, prostate cancer cell line, see, e.g., Korenchuk et al., In Vivo, V. 15, pp. 163-168 (2001)) were inoculated directly into the tibias of immunocompromised mice (n=8). Bony lesions developed in 8/8 animals with an osteoblastic bone response that mimicked the situation in human prostate cancer. In addition, 8/8 bone lesions stained positively for Prostatic Acid Phosphatase, with only heterogeneous AR and PSA immunoreactivity (see FIG. 3). The PAP expression was strongly and uniformly positive in all samples tested. These results are consistent with the results obtained for human prostate cancer bone metastases samples (see FIG. 2).

### Example 5

### Effect of Tartrate on VCaP Prostate Cancer Cells

The human prostate cancer cell line VCaP (originally derived from a vertebral metastases) and the pre-osteoblast cell line MC3T3, were used to for in vitro studies with tartrate. Each cell culture was grown in serum free medium either in single line or co-culture, and treated with or without tartrate (20 μM) for 7 days. Cell numbers were counted by a hemacytometer and the secretion of PAP and bone alkaline phosphatase (ALP) was measured using ELISA.

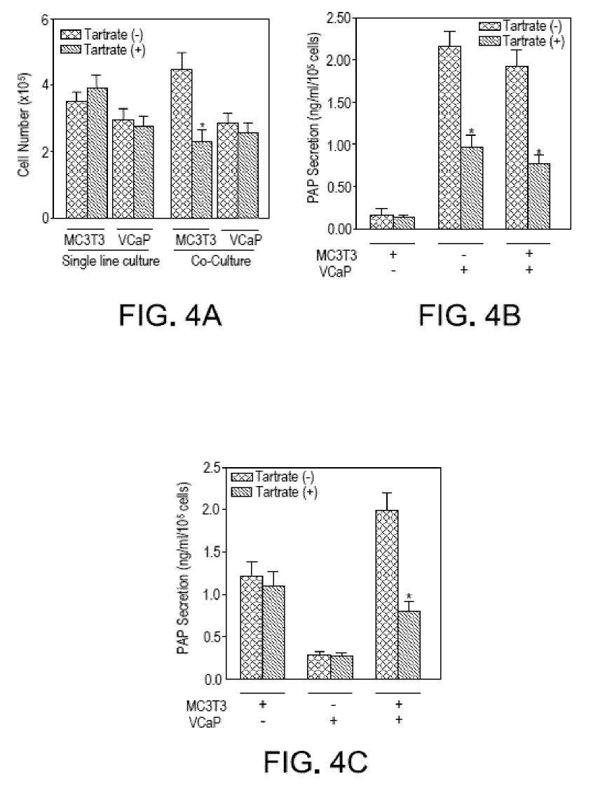

FIG. 4A shows the results of tartrate addition to the growth of MC3T3 and VCaP single line cell cultures, as well as MC3T3 and VCaP co-cultures. The figure demonstrates that tartrate addition did not have a significant effect on the growth of VCaP or MC3T3 single line cell cultures, or VCaP cell growth, when co-cultured with MC3T3 cells. However, tartrate significantly inhibited the growth of MC3T3 cells, when co-cultured with VCaP cells (p<0.01, see FIG. 4A). These results indicate that PAP inhibition by tartrate serves to attenuate the growth of osteoblast cells which may interrupt bone metastases.

The ability of tartrate to inhibit the secretion of prostatic acid phosphatase (PAP) from MC3T3 and VCaP single line and co-cultures was also measured. As shown in FIG. 4B, tartrate addition significantly inhibited prostatic acid phosphatase secretion by VCaP cells grown alone or in co-culture with MC3T3 cells. PAP was secreted at very low levels by MC3T3 cells, and tartrate did not inhibit this secretion (FIG. 4B).

Secretion of bone alkaline phosphatase (ALP) by pre-osteoblast cells is correlated with pre-osteoblast differentiation into mature osteoblasts cells. Accordingly, ALP secretion was measured to indirectly determine whether the inhibition of PAP secretion by tartrate also served to attenuate differentiation events. FIG. 4C demonstrates that tartrate addition significantly inhibited bone alkaline phosphatase secretion by MC3T3 pre-osteoblast cells when grown in co-culture with VCaP cells. This effect was not seen in the single line MC3T3 and VCaP cell cultures.

Taken together, the data described in FIG. 4 demonstrates that tartrate inhibits PAP secretion by VCaP prostate cancer cells, which abrogates their stimulatory effects on (1) bone cell growth (osteoblast growth) and (2) alkaline phosphatase production.

### Example 6

### Bisphosphonate-Tartrate and Bisphosphonate-Glyceric Acid Conjugates

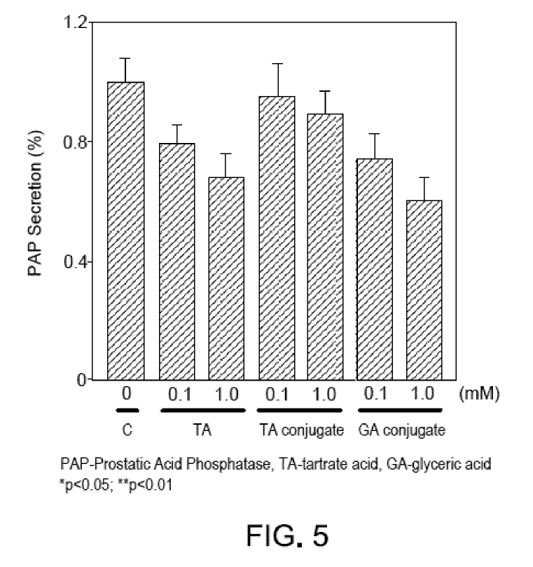

The compounds synthesized in examples 1 and 2 were tested for their ability to inhibit PAP secretion. Either the glyceric acid conjugate (i.e., N-Alendronyl-D-Glyceramide) (example 1, 0.1 mM or 1.0 mM) or the tartaric acid conjugate (i.e., N-Alendronyl-L-Tartaric Acid Monamide) (example 2, 0.1 mM or 1.0 mM) was added to VCaP cell culture medium, and incubated overnight. PAP secretion was measured using an ELISA assay. The results of these experiments are given in FIG. 5. The figure shows that both compounds served to inhibit PAP secretion, at all concentrations tested. The results with the glyceric acid conjugate were more pronounced, with PAP secretion significantly inhibited at both concentrations tested (FIG. 5). Additionally, the glyceric acid conjugate was as effective as tartrate alone, as a PAP inhibitor.

### Example 7

### Determination of the Effects of PAP on Osteoclast Differentiation and Activity

All cell lines were purchased from American Type Culture Collection (ATCC), Rockville, Md., media from Life Technologies, Grand Island, N.Y., reagents from Sigma, St. Louis, Mo., and 12-well co-culture plates and inserts with 0.4 μm pores in addition to BD BioCoat™ Osteologic™ Bone Cell Culture System from BD Inc, Bedford, Mass.

In order to determine the effects of prostatic acid phosphatase (PAP) on osteoclast differentiation and activity, a series of co-culture experiments were performed in which pre-osteoclast cells (RAW cells) were co-cultured with either PAP-positive (PAP⁺), VCaP cells or PAP-negative (PAP⁻) (PC3) human prostate cancer (PCa) cells. RAW cells were counted in a hemacytometer on day 5.

VCaP (PAP⁺) and PC3 (PAP⁻) cells (5×10⁴ each) were seeded on inserts in RPMI or DMEM medium with 10% FBS. Pre-osteoclast, PRAW cells (5×10⁴) were plated on the bottom in 12 well plates. After 2 days, medium was changed to serum-free DMEM with 0.1% BSA+/− tartrate (2004) for 2 days. Mean number of multinucleated cells (hallmark of osteoclast differentiation) was determined on day 5 by multiple field counts in 200× objective view.

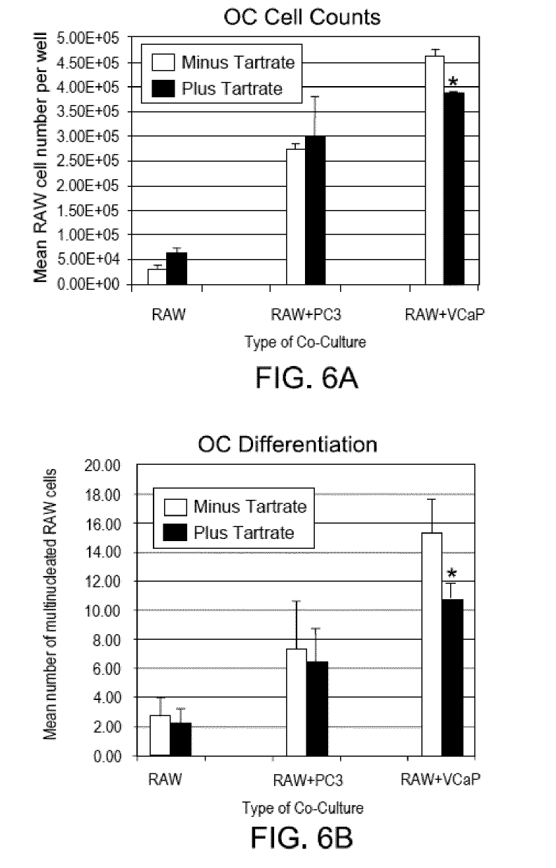

Tartrate addition did not have a significant effect on the growth of RAW cells alone or co-cultured with PC3 cells, but significantly inhibited the growth of RAW cells when co-cultured with VCaP cells (see FIG. 6A), suggesting that PAP secreted by VCaP cells stimulated RAW cell growth (*p<0.01).

Tartrate did not have a significant effect on differentiation of RAW cells alone or co-cultured with PC3 cells, but significantly inhibited RAW cell differentiation when co-cultured with VCaP (see FIG. 6B), indicating that PAP secreted by VCaP cells stimulated RAW cell differentiation (*p<0.01).

The effect of PAP on osteoclast bone-resorbing activity was measured by assaying pit formation when osteoclasts were cultured on bone matrix (osteologic discs). RAW (osteoclast) cells (2×10⁴ cells), PC3 (PAP⁻) cells (2×10⁴ cells), and VCaP (PAP⁺) cells (3×10⁴ cells) were plated, alone or in combination, on calcium hydroxyapatite-coated osteologic discs in DMEM medium with 10% FBS for 7 days+/−tartrate (20 μM). All cultures were treated with RANKL (50 ng/μl) and MCSF (25 ng/μl). On day 7, cells were washed and discs stained with von Kossa to visualize the calcium matrix and pits (which indicate bone resorption by the RAW osteoclast cells). Culturing of RAW osteoclast cells alone resulted in a low number of pits. Respective culturing of PC3 and VCAP cells alone resulted in essentially no pits. Co-culturing of RAW cells and PC3 cells resulted in a low number of pits and tartrate addition had no significant effect on the number of pits. Strikingly, co-culturing of RAW cells and VCaP make resulted in formation of numerous pits (10-fold more than RAW alone) and addition of tartrate significantly inhibited pit formation (decreased by 50%). These results demonstrated that PAP secreted by VCaP cells enhanced osteoclast resorptive activity.

The above experimental results demonstrate that PAP derived from PCa cells stimulates osteoclast growth, differentiation and activity in terms of ability to resorb bone matrix. Specifically, PAP secreted by the human PCa cell line VCaP increased both the differentiation and bone-resorbing activity of pre-osteoclast cells and these effects were diminished by the addition of a small-molecule inhibitor of PAP enzymatic activity, L-tartrate. This further supports the idea that inhibition of PAP can prevent and/or treat prostate cancer bone metastases by inhibiting osteoclastic bone-resorption and the subsequent release of growth-promoting factors from bone matrix.

All patents, patent applications, publications, product descriptions, and protocols which are cited throughout this application are incorporated herein by reference in their entireties. The embodiments illustrated and discussed in this specification are intended only to teach those skilled in the art the best way known to the inventors to make and use the invention. Nothing in this specification should be considered as limiting the scope of the present invention. Modifications and variation of the above-described embodiments of the invention are possible without departing from the invention, as appreciated by those skilled in the art in light of the above teachings. It is therefore understood that, within the scope of the claims and their equivalents, the invention may be practiced otherwise than as specifically described.

What is claimed:

1. A compound comprising a bisphosphonate covalently bonded to a prostatic acid phosphatase (PAP) inhibitor, or a pharmaceutically acceptable salt thereof.

2. The compound of claim 1, wherein the bisphosphonate is alendronate.

3. The compound of claim 1, wherein the prostatic acid phosphatase (PAP) inhibitor is tartrate or glyceric acid.

4. The compound of claim 1 that is N-Alendronyl-L-Tartaric Acid Monamide.

5. The compound of claim 1 that is N-Alendronyl-D-Glyceramide.

6. A composition comprising the compound of claim 1 and a pharmaceutically acceptable excipient.

7. A method for inhibiting prostate cancer bone metastasis in a subject in need thereof, comprising, administering to the subject an effective amount of the compound of claim 1.

8. The method of claim 7, comprising administering the compound in a composition comprising a pharmaceutically acceptable excipient.

9. The method of claim 8, wherein the method comprises oral administration of the composition.

10. The method of claim 8, wherein the method comprises parenteral administration of the composition.

11. A method for treating prostate cancer bone metastasis in a subject in need thereof, comprising: administering to the subject an effective amount of the compound of claim 1.

12. The method of claim 11, comprising administering the compound in a composition comprising a pharmaceutically acceptable excipient.

13. The method of claim 12, wherein the method comprises oral administration of the composition.

14. The method of claim 12, wherein the method comprises parenteral administration of the composition.

15. A method for inhibiting the activity of PAP in a subject in need thereof, comprising administering to the subject, an effective amount of the compound of claim 1.

16. The method of claim 15, comprising administering the compound in a composition comprising a pharmaceutically acceptable excipient.

17. A method for making a PAP inhibitor orally active, comprising covalently bonding the PAP inhibitor to a bisphosphonate.

18. The method of claim 17, wherein the bisphosphonate is alendronate and the PAP inhibitor is selected from tartaric acid and glyceric acid.
